(19) United States
(12) Patent Application Publication
(10) Pub. No.: US 2014/0378545 A1
(43) Pub. Date: Dec. 25, 2014

(54) PREGABALIN GR TABLETS

(71) Applicant: RANBAXY LABORATORIES LIMITED, New Delhi, Delhi (IN)

(72) Inventors: Varinder Kumar, Mohali (IN); Shavej Ahmad, Lucknow (IN); Romi Barat Singh, Varanasi (IN)

(21) Appl. No.: 14/374,500

(22) PCT Filed: Jan. 29, 2013
(86) PCT No.: PCT/IB2013/050762

§ 371 Date: Jul. 24, 2014

(30) Foreign Application Priority Data
Jan. 30, 2012 (IN) 249/DEL/2012

(51) Int. Cl. A61K 9/20; A61K 31/197

(52) U.S. Cl. CPC A61K 9/2027; A61K 31/197; A61K 9/2031
USPC 514/561

(57) ABSTRACT

The present invention relates to a gastroretentive tablet comprising pregabalin, an acrylic acid polymer, one or more swellable polymers, and other pharmaceutically acceptable excipients. It further relates to a process for the preparation of same.

### FIELD OF THE INVENTION

The present invention relates to a gastroretentive tablet comprising pregabalin, an acrylic acid polymer, one or more swellable polymers, and other pharmaceutically acceptable excipients. It further relates to a process for the preparation of same.

### BACKGROUND OF THE INVENTION

Pregabalin, or (S)-3-(aminomethyl)-5-methylhexanoic acid, binds to the calcium channel alpha-2-delta (α2δ) subunit and is related to endogenous inhibitory neurotransmitter gamma-amino butyric acid (GABA), which is involved in brain neuronal activity. In the United States, pregabalin has been approved for the management of neuropathic pain associated with diabetic peripheral neuropathy, management of post herpetic neuralgia, management of fibromyalgia, and as an adjunctive therapy for adult patients with partial onset seizures.

Pregabalin is disclosed in U.S. Pat. Nos. 6,197,819 and 5,563,175, which describe the use of pregabalin in the treatment of seizure disorders. U.S. Pat. No. 6,117,906 discloses the use of pregabalin in treating anxiety, while U.S. Pat. No. 6,001,876 discloses its use in treating pain.

Currently, pregabalin is available as conventional immediate-release capsules marketed by CP Pharms/Pfizer, under the brand name Lyrica®, and requires two or three times a day dosing. The importance of taking drugs at regular intervals cannot be overemphasized. However, it is not easy for everyone to remember to take the correct dose at the same time each day. Multiple dosing is not only inconvenient, but it also lowers patient compliance. Once daily dosing generally improves patient compliance as well as reduces the severity and frequency of side effects by reducing peak blood levels, and may also increase drug efficacy by increasing minimum plasma concentration. Once daily dosing of pregabalin, however, presents numerous challenges. Conventional extended-release compositions are problematic as pregabalin does not have uniform absorption throughout the gastrointestinal tract. Pregabalin is absorbed well in the small intestine and the ascending colon, but is poorly absorbed beyond the hepatic flexure. This suggests that the mean absorption window for pregabalin is, on average, about six hours or less and any drug release from a conventional extended-release dosage form beyond six hours would thus be wasted because the dosage form has travelled beyond the hepatic flexure.

U.S. Patent Application No. 2007/0269511 discloses a pregabalin formulation containing a matrix forming agent and a swelling agent, wherein the matrix forming agent is polyvinyl acetate and polyvinylpyrrolidone, and the swelling agent is cross-linked polyvinylpyrrolidone. U.S. Patent Application No. 2011/0135723 describes once-daily pharmaceutical compositions of pregabalin wherein the excipients include one or more water-insoluble components or a combination of one or more water-insoluble components and one or more water-soluble components. U.S. Patent Application No. 2010/0255067 describes pharmaceutical compositions comprising pregabalin, a hydrophobic release controlling agent, and other pharmaceutically acceptable excipients. PCT Publication No. WO 2011/151708 describes a gastroretentive dosage form comprising a GABA analog, at least one swelling agent, and at least one non-swelling release retardant.

Therefore, a sustained-release gastroretentive dosage form would be an ideal dosage form for drug candidates like pregabalin. The objective of the present invention is to develop a gastroretentive tablet of pregabalin that not only extends the release of pregabalin but also retains pregabalin in the upper parts of the gastrointestinal tract for a long period of time to overcome its decreased colonic absorption.

### SUMMARY OF THE INVENTION

In one general aspect, the invention relates to a gastroretentive tablet comprising pregabalin, an acrylic acid polymer, one or more swellable polymers, and other pharmaceutically acceptable excipients.

In an embodiment of the above aspect, the gastroretentive tablet may comprise pregabalin, an acrylic acid polymer, and one or more swellable polymers selected from polyethylene oxide, hydroxypropylmethylcellulose, cross linked polyvinylpyrrolidone, and combinations thereof.

In another embodiment, the other pharmaceutically acceptable excipients are selected from diluents, binders, disintegrants, glidants, lubricants, and coloring agents.

In another general aspect, it relates to a process for the preparation of a gastroretentive tablet comprising pregabalin, an acrylic acid polymer, one or more swellable polymers, and other pharmaceutically acceptable excipients selected from diluents, binders, disintegrants, glidants, lubricants, and coloring agents, wherein the process comprises the conventional methods of dry granulation, wet granulation or direct compression.

### DETAILED DESCRIPTION OF THE INVENTION

“Pregabalin”, as recited herein, means pregabalin or a pharmaceutically acceptable form of pregabalin, including without limitation, its free form (zwitterion) and its pharmaceutically acceptable complexes, salts, enantiomers, solvates, hydrates, and polymorphs.

One of the approaches that can be used for achieving gastric retention involves the use of swelling and expanding systems. These systems are usually monolithic tablets and are comprised of the drug and one or more swellable polymers. These polymers swell unrestrained via imbibition of gastric fluid to such an extent that it causes the tablet to float on gastric contents. The air entrapped by the swollen polymer confers buoyancy to these tablets. For an ideal gastroretentive effect, the polymers selected should be such that they swell in contact with gastric fluid as well as sufficiently reduce the density of the tablet. The tablets of the present invention utilize the combination of an acrylic acid polymer and one or more swellable polymers. Acrylic acid polymer (Acritamer®/Carbopol®), also known variously as carbomer, polyacrylic acid, carboxyvinyl polymer, or carboxy polymethylene, is a synthetic high molecular weight polymer of acrylic acid that is cross-linked with either allyl sucrose or allyl ethers of pentaerythritol. They swell in water to form a gel when exposed to a pH environment above 4.0 to 6.0. In the gastroretentive tablets described herein, the addition of carbomer extends the rate of release of pregabalin and simultaneously causes floating of the tablet on the gastric contents owing to its low density.

The swellable polymer(s), as recited herein, include polyalkylene oxides, preferably polyethylene oxide available under the trade name Polyox™; polyethylene oxide-polypropylene oxide block copolymers available under the trade names Pluronic® and Tectonic™; cellulosic polymers such as methylcellulose, hyrdoxymethylcellulose, hydroxyethylcellulose, hydroxypropylcellulose, hydroxypropylmethylcellulose, ethyl cellulose, calcium carboxymethylcellulose, or sodium carboxymethylcellulose; a vinyl pyrrolidone polymer such as crosslinked polyvinylpyrrolidone or crospovidone; copolymers of vinyl pyrrolidone and vinyl acetate; polysaccharides such as starch and starch-based polymers, chitosan, agar, alginates, carrageenan, furcellaran, guar gum, gum arabic, gum tragacanth, karaya gum, locust bean gum, pectin, dextran, gellan gum, rhamsan gum, welan gum, xanthan gum, propylene glycol alginate, or hydroxypropyl guar; and combinations thereof. Particularly preferred among these are polyethylene oxide, hydroxypropylmethylcellulose, crosslinked polyvinylpyrrolidone, and the combinations thereof.

The tablets may contain other pharmaceutically acceptable excipients that are routinely used and may be selected from diluents, binders, disintegrants, glidants, lubricants, coloring agents, and mixtures thereof.

Exemplary diluents may include, but are not limited to, microcrystalline cellulose, silicified microcrystalline cellulose, microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, or combinations thereof.

Exemplary binders may include, but are not limited to, acacia, guar gum, alginic acid, carbomer, dextrin, maltodextrin, methylcellulose, ethyl cellulose, hydroxyethylcellulose, hydroxypropylcellulose, hydroxypropylmethylcellulose, carboxymethylcellulose sodium, magnesium aluminum silicate, polymethacrylates, crospovidones, povidones, copovidones, gelatin, starch, or combinations thereof.

Exemplary disintegrants include, but are not limited to, mannitol, alginic acid, carboxymethylcellulose, hydroxypropylcellulose, microcrystalline cellulose, croscarmellose sodium, crospovidone, magnesium aluminum silicate, methylcellulose, povidone, sodium alginate, sodium starch glycolate, starch, or combinations thereof.

Exemplary lubricants/glidants include, but are not limited to, magnesium stearate, zinc stearate, calcium stearate, stearic acid, colloidal silicon dioxide, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxyethylene monostearate, cocoa butter, hydrogenated vegetable oils, mineral oil, polysaccharides, or combinations thereof.

Exemplary coloring agents include, but are not limited to, titanium dioxide pigments, lake colors, iron oxide pigments, or combinations thereof.

The tablets prepared may further be optionally coated. Coatings may be employed for aesthetic purpose or for stabilizing the tablets or for retarding the drug-release. The coating may be carried out using conventional techniques employing conventional ingredients. For example, the tablets may be coated with one of the commercially available coating systems or any one of polymeric film coatings routinely used, such as ethyl cellulose, hydroxypropylmethylcellulose, hydroxypropylcellulose, methylcellulose, carboxymethyl cellulose, hydroxyl methylcellulose, cellulose acetate, waxes such as polyethylene glycol, methacrylic acid polymers, and the like.

The tablets described herein may be prepared by conventional processes using commonly available equipments. The process may comprise wet granulation, dry granulation, or direct compression processes.

The gastroretentive tablets of pregabalin, as described herein, may take the form of several different embodiments.

In one embodiment, the gastroretentive tablet comprises pregabalin, a polymer system comprising Carbopol®, polyethylene oxide, cross-linked polyvinylpyrrolidone, and other pharmaceutically acceptable excipients.

In another embodiment, the gastroretentive tablet comprises pregabalin, a polymer system comprising Carbopol®, hydroxypropylmethylcellulose, and other pharmaceutically acceptable excipients.

In another embodiment, the gastroretentive tablet comprises pregabalin, a polymer system comprising Carbopol®, hydroxypropylmethylcellulose, cross-linked polyvinylpyrrolidone, and other pharmaceutically acceptable excipients.

In another embodiment, it relates to process of preparing a gastroretentive tablet comprising pregabalin, a polymer system comprising of Carbopol®, swellable polymer(s), and other pharmaceutically acceptable excipients wherein the process comprises the steps of:
a) sifting pregabalin, Carbopol®, swellable polymer(s) and other pharmaceutically acceptable excipients through a suitable sieve and thoroughly blending for a desired time;
b) sifting magnesium stearate through a suitable sieve;
c) blending the material of step a) with the material of step b) for a suitable time; and
d) compressing the lubricated blend of step c) into tablets using appropriate tooling.

From the above it is apparent that various modifications and combinations of the formulations detailed in the text may be made without departing from the spirit and scope of the invention. The invention as described herein may be illustrated by the following examples but is not to be construed to be limited by them.

### EXAMPLES 1-4

| | Quantity (mg/tablet) | | | |
|---|---|---|---|---|
| Ingredients | Example 1 | Example 2 | Example 3 | Example 4 |
| Pregabalin | 330.00 | 330.00 | 330.00 | 330.00 |
| Carbopol ® | 210.00 | 160.00 | 90.00 | 95.00 |
| Polyethylene oxide | 200.00 | 250.00 | 320.00 | 365.00 |
| Crospovidone | 250.00 | 250.00 | 250.00 | 200.00 |
| Magnesium stearate | 10.00 | 10.00 | 10.00 | 10.00 |
| Total weight | 1000.00 | 1000.00 | 1000.00 | 1000.00 |

### Procedure:

a) Pregabalin, Carbopol®, polyethylene oxide and crospovidone were sifted through a suitable sieve and thoroughly blended for a desired time;
b) Magnesium stearate was separately sifted through a suitable sieve;
c) Material of step a) was blended with the material of step b) for a suitable time;
d) The lubricated blend of step c) was compressed into tablets using appropriate tooling.

The tablets thus obtained were subjected to dissolution testing at 37° C. using United States Pharmacopoeia Type II (paddle) dissolution apparatus at 50 rpm. The dissolution medium used was 900 ml of 0.06N HCl. The results of the dissolution test are recorded in Table 1 below.

TABLE 1

| | |
|---|---|
| Time | Percentage of Drug Released |

| (hours) | Example 1 | Example 2 | Example 3 | Example 4 |
|---|---|---|---|---|
| 1 | 16 | 15 | 14 | 15 |
| 2 | 25 | 24 | 24 | 24 |
| 4 | 38 | 37 | 38 | 39 |
| 6 | 49 | 48 | 49 | 50 |
| 9 | 61 | 60 | 64 | 63 |
| 12 | 74 | 72 | 76 | 73 |
| 16 | 84 | 81 | 86 | 89 |
| 20 | 95 | 93 | 96 | 95 |
| 24 | 100 | 99 | 101 | 100 |

### EXAMPLES 5-8

| | Quantity (mg/tablet) | | | |
|---|---|---|---|---|
| Ingredients | Example 5 | Example 6 | Example 7 | Example 8 |
| Pregabalin | 330.00 | 330.00 | 330.00 | 330.00 |
| Carbopol ® | 90.00 | 90.00 | 90.00 | 90.00 |
| Crospovidone | 250.00 | 250.00 | — | — |
| Methocel ™ K100 LV | 320.00 | — | — | — |
| CR | | | | |
| Methocel ™ E50 | — | 80.00 | 240.00 | 283.00 |
| Methocel ™ K4M | — | 240.00 | 320.00 | 277.00 |
| Silicon dioxide | — | — | 10.00 | 10.00 |
| Magnesium stearate | 10.00 | 10.00 | 10.00 | 10.00 |
| Total weight | 1000.00 | 1000.00 | 1000.00 | 1000.00 |

### Procedure:

a) Pregabalin, Carbopol®, swellable polymer(s) and other pharmaceutically acceptable excipients were sifted through a suitable sieve and thoroughly blended for a desired time;
b) Magnesium stearate was separately sifted through a suitable sieve;
c) Material of step a) was blended with the material of step b) for a suitable time; and
d) The lubricated blend of step c) was compressed into tablets using appropriate tooling.

The tablets thus obtained were subjected to dissolution testing at 37° C. using United States Pharmacopoeia Type II (paddle) dissolution apparatus at 50 rpm. The dissolution medium used was 900 ml of 0.06N HCl. The results of the dissolution test are recorded in Table 2 below.

TABLE 2

| | |
|---|---|
| Time | Percentage of Drug Released |

| (hours) | Example 5 | Example 6 | Example 7 | Example 8 |
|---|---|---|---|---|
| 1 | 18 | 18 | 15 | 15 |
| 2 | 28 | 28 | 24 | 23 |
| 4 | 42 | 42 | 36 | 35 |
| 6 | 53 | 54 | 45 | 45 |
| 9 | 65 | 68 | 57 | 58 |
| 12 | 76 | 78 | 69 | 68 |
| 16 | 86 | 88 | 78 | 79 |
| 20 | 92 | 96 | 87 | 89 |
| 24 | 98 | 101 | 93 | 93 |

We claim:

1. A gastroretentive tablet comprising pregabalin, an acrylic acid polymer, one or more swellable polymers, and other pharmaceutically acceptable excipients.

2. The gastroretentive tablet according to claim 1, wherein the swellable polymer is selected from the group consisting of polyethylene oxide, hydroxypropylmethylcellulose, cross linked polyvinylpyrrolidone, and combinations thereof.

3. The gastroretentive tablet according to claim 1, wherein the other pharmaceutically acceptable excipients are selected from diluents, binders, disintegrants, glidants, lubricants, and coloring agents.

4. A process for the preparation of a gastroretentive tablet comprising pregabalin, an acrylic acid polymer, one or more swellable polymers, and other pharmaceutically acceptable excipients selected from diluents, binders, disintegrants, glidants, lubricants, and coloring agents, wherein the process comprises the conventional methods of dry granulation, wet granulation, or direct compression.
